(19) United States
(12) Patent Application Publication
(10) Pub. No.: US 2011/0129873 A1
(43) Pub. Date: Jun. 2, 2011

(54) Recombinant DNA Vectors for Expression of Human Prolactin Antagonists

(75) Inventors: Gregg Bogosian, St. Louis, MO (US); Patricia Morris, St. Louis, MO (US); Jennifer Chou, St. Louis, MO (US); Kimberly Allen, St. Louis, MO (US); Julia Frantz, St. Louis, MO (US); Brad Storrs, St. Louis, MO (US); Jacob Tou, St. Louis, MO (US)

(73) Assignee: MONSANTO TECHNOLOGY LLC, St. Louis, MO (US)

(21) Appl. No.: 12/990,142

(22) PCT Filed: Apr. 30, 2009
(86) PCT No.: PCT/US09/42393

§ 371 Date: Feb. 8, 2011

Related U.S. Application Data
(60) Provisional application No. 61/049,122, filed on Apr. 30, 2008.
(60) Provisional application No. 61/050,392, filed on May 5, 2008.
(60) Provisional application No. 61/090,634, filed on Aug. 21, 2008.
(60) Provisional application No. 61/138,673, filed on Dec. 18, 2008.

(51) Int. Cl. C12N 15/70; C12N 1/21; C12P 21/02; C07H 21/00

(52) U.S. Cl. 435/69.1; 435/320.1; 435/243; 536/23.1

(57) ABSTRACT

Embodiments of the present invention relate generally to methods and compositions for producing human prolactin antagonists. Embodiments of the present invention also relate to various methods for improved production of human prolactin antagonists by microorganisms. These microorganisms, including Escherichia coli, can be transformed to enable the expression of human prolactin antagonists at high levels using conventional and inexpensive fermentation conditions and inexpensive induction conditions, as described herein.

### FIELD OF THE INVENTION

This invention relates generally to methods and compositions for producing human prolactin antagonists. More specifically, it relates to various methods for improved production of human prolactin antagonists by microorganisms, to improved methods for the recovery and purification of such human prolactin antagonists, and to the use of human prolactin antagonists prepared by these methods for use in the treatment of human diseases, including use in treatment of cancers.

### BACKGROUND

Prolactin is a protein hormone that is structurally related to growth hormone. Prolactin acts on cells with prolactin receptors and is required for the proliferation and differentiation of such cells. Associations have been made between prolactin activity and cancers involving cell types that have prolactin receptors, including breast cancer and prostate cancer, wherein prolactin has been shown to promote the proliferation of the cancerous cell types. Thus, it has been proposed that interventions involving disruption of the normal prolactin interaction with its receptor may be useful in the treatment of cancers including breast cancer and prostate cancer wherein the cell types have prolactin receptors. References describing the use of human prolactin antagonists include U.S. Pat. No. 7,115,556, U.S. Pat. No. 7,201,905, U.S. Pat. No. 7,339,027, and European Patent EP 1079851 B1, each of which is hereby incorporated herein by reference in its entirety.

A variant form of human prolactin, having the glycine amino acid residue at position 129 of the prolactin protein chain replaced by the amino acid arginine (the G129R variant) is known to be a potent antagonist of prolactin. The G129R human prolactin antagonist has been found to inhibit the cell proliferation-promoting effects of prolactin on its receptor. For example, U.S. Pat. No. 7,115,556 describes the G129R human prolactin variant as an antagonist of wild-type human prolactin at the prolactin receptor, and for the use of such a human prolactin antagonist to inhibit the proliferation of cells with prolactin receptors. Such a human prolactin antagonist may be useful in the treatment of diseases involving uncontrolled cell proliferation, including breast cancer and prostate cancer. Other human prolactin antagonists may also be useful as therapeutic agents; however, substantial amounts of human prolactin antagonists would need to be produced to treat human diseases such as breast cancer and prostate cancer.

While important applications have previously been identified, it is believed that prior attempts to produce prolactin antagonists have not been economically feasible due to relatively inefficient and expensive means for expressing, recovering and purifying these proteins. Therefore, there exists a substantial need for the efficient and inexpensive production of human prolactin antagonists.

### SUMMARY OF THE INVENTION

Described herein is a family of human prolactin antagonist expression vectors with which microorganisms can be transformed to enable the expression of human prolactin antagonists at high levels using conventional and inexpensive fermentation conditions and inexpensive induction conditions. This family is exemplified by the 25 vectors listed in Table 2 each of which comprises the corresponding sequence of SEQ ID NOs: 1 through 23 plus SEQ ID NOs: 54 through 55, as indicated in Table 2. Each of SEQ ID NOs: 1 through 23 extends from an EcoRI site (GAATTC) before the promoter region, through the promoter, ribosome binding site (“RBS”), the entire human prolactin antagonist structural gene, and a transcription terminator, ending with a HindIII site (AAGCTT). Each of SEQ ID NOs: 54 through 55 extends from a ClaI site (ATCGAT) before a gene encoding a repressor protein that regulates the promoter, followed by the promoter region, through the promoter, ribosome binding site (“RBS”), the entire human prolactin antagonist structural gene, and a transcription terminator, ending with a SalI site (GTCGAC). Methods have also been developed to use these vectors to transform Escherichia coli and thereby efficiently and economically produce human prolactin antagonist.

### DETAILED DESCRIPTION OF THE INVENTION

The present invention is based in part on the observation that the efficient and economical production of human prolactin has generally been unobtainable. Surprisingly, it has been discovered that use of a unique ribosome binding site (“RBS”), a unique, synthetic front end of a prolactin gene, or a combination of both a synthetic RBS and a synthetic front end, and in particular, a synthetic front end of a human prolactin gene, can be used to form expression vectors that allow for the efficient and inexpensive production of human prolactin and human prolactin variants, such as prolactin antagonists, in microorganisms. As further described below, embodiments of the present invention relate to various methods for improved production of human prolactin antagonists by microorganisms. More particularly, Applicants have discovered a family of human prolactin antagonist expression vectors for transforming microorganisms to enable the expression of human prolactin antagonists at high levels using conventional and inexpensive fermentation conditions and inexpensive induction conditions.

Thus, one aspect of the present invention is an expression vector or family of expression vectors for transforming microorganisms to enable the expression of human prolactin antagonists. The family of expression vectors is exemplified by the 25 vectors listed in Table 2 each of which comprises the corresponding sequence of SEQ ID NOs: 1 through 23 plus SEQ ID NOs: 54 through 55, as indicated in Table 2. Each of SEQ ID NOs: 1 through 23 extends from an EcoRI site (GAATTC) before the promoter region, through the promoter, ribosome binding site (“RBS”), the entire human prolactin antagonist structural gene, and a transcription terminator, ending with a HindIII site (AAGCTT). Each of SEQ ID NOs: 54 through 55 extends from a ClaI site (ATCGAT) before a gene encoding a repressor protein that regulates the promoter, followed by the promoter region, through the promoter, ribosome binding site (“RBS”), the entire human prolactin antagonist structural gene, and a transcription terminator, ending with a SalI site (GTCGAC). The expression vectors can transform microorganisms, including Escherichia coli, to enable the expression of human prolactin antagonists at high levels using conventional and inexpensive fermentation conditions and inexpensive induction conditions.

Another aspect of the invention includes recombinant constructs comprising a ribosome binding site operably linked to a synthetic front end of a prolactin gene. As used herein, the term “operably linked” refers to nucleic acid sequences that are placed in a functional relationship with other nucleic acid sequences. For example, a promoter is operably linked to a ribosome binding site and a coding sequence if it effects the transcription of the ribosome binding site and coding sequence. A ribosome binding site is operably linked to a coding sequence if it effects the translation of the coding sequence. A synthetic front end of a coding sequence is operably linked to the remainder of the coding sequence if the two coding sequences are in the same reading frame and effect the translation of the coding sequence into the desired protein. A ribosome binding site is operably linked to a synthetic front end of a coding sequence if the combination effects translation of the synthetic front end of the coding sequence operably linked to the remainder of the coding sequence.

Thus, in one embodiment, the recombinant construct comprises a ribosome binding site selected from the group consisting of SEQ ID NOS: 24-30, 56, and 57 operably linked to a synthetic front end of a prolactin gene. In another embodiment, the recombinant construct comprises a ribosome binding site selected from the group consisting of SEQ ID NOs: 24-30, 56, and 57 operably linked to a synthetic front end of a prolactin gene selected from the group consisting of SEQ ID NOs: 31-36. Accordingly, by way of example, embodiments of the recombinant constructs comprising a synthetic ribosome binding site operably linked to a synthetic front end of a prolactin gene as listed in Table 1 may be achieved. In certain embodiments, any one of these constructs may also comprise a prolactin antagonist DNA sequence wherein the ribosome binding site, synthetic front end of a prolactin gene and the prolactin agonist DNA sequence are operably linked. These components may also be operably linked to a promoter. In preferred embodiments, the prolactin antagonist DNA sequence encodes the G129R variant human prolactin antagonist as disclosed, for example, in U.S. Pat. No. 7,115,556.

TABLE 1
Combinations of a synthetic ribosome binding sitesoperably
linked to synthetic front end of a prolactin gene

| Ribosome Binding Site | Synthetic Front End of Prolactin Gene |
|---|---|
| SEQ ID NO: 24 | SEQ ID NO: 31 |
| SEQ ID NO: 24 | SEQ ID NO: 32 |
| SEQ ID NO: 24 | SEQ ID NO: 33 |
| SEQ ID NO: 24 | SEQ ID NO: 34 |
| SEQ ID NO: 24 | SEQ ID NO: 35 |
| SEQ ID NO: 24 | SEQ ID NO: 36 |
| SEQ ID NO: 24 | SEQ ID NO: 37 |
| SEQ ID NO: 24 | SEQ ID NO: 38 |
| SEQ ID NO: 24 | SEQ ID NO: 39 |
| SEQ ID NO: 24 | SEQ ID NO: 40 |
| SEQ ID NO: 24 | SEQ ID NO: 41 |
| SEQ ID NO: 24 | SEQ ID NO: 42 |
| SEQ ID NO: 24 | SEQ ID NO: 43 |
| SEQ ID NO: 24 | SEQ ID NO: 44 |
| SEQ ID NO: 24 | SEQ ID NO: 45 |
| SEQ ID NO: 24 | SEQ ID NO: 46 |
| SEQ ID NO: 24 | SEQ ID NO: 47 |
| SEQ ID NO: 24 | SEQ ID NO: 48 |
| SEQ ID NO: 24 | SEQ ID NO: 49 |
| SEQ ID NO: 24 | SEQ ID NO: 50 |
| SEQ ID NO: 24 | SEQ ID NO: 51 |
| SEQ ID NO: 24 | SEQ ID NO: 52 |
| SEQ ID NO: 24 | SEQ ID NO: 53 |
| SEQ ID NO: 24 | SEQ ID NO: 58 |
| SEQ ID NO: 25 | SEQ ID NO: 31 |
| SEQ ID NO: 25 | SEQ ID NO: 32 |
| SEQ ID NO: 25 | SEQ ID NO: 33 |
| SEQ ID NO: 25 | SEQ ID NO: 34 |
| SEQ ID NO: 25 | SEQ ID NO: 35 |
| SEQ ID NO: 25 | SEQ ID NO: 36 |
| SEQ ID NO: 25 | SEQ ID NO: 37 |
| SEQ ID NO: 25 | SEQ ID NO: 38 |
| SEQ ID NO: 25 | SEQ ID NO: 39 |
| SEQ ID NO: 25 | SEQ ID NO: 40 |
| SEQ ID NO: 25 | SEQ ID NO: 41 |
| SEQ ID NO: 25 | SEQ ID NO: 42 |
| SEQ ID NO: 25 | SEQ ID NO: 43 |
| SEQ ID NO: 25 | SEQ ID NO: 44 |
| SEQ ID NO: 25 | SEQ ID NO: 45 |
| SEQ ID NO: 25 | SEQ ID NO: 46 |
| SEQ ID NO: 25 | SEQ ID NO: 47 |
| SEQ ID NO: 25 | SEQ ID NO: 48 |
| SEQ ID NO: 25 | SEQ ID NO: 49 |
| SEQ ID NO: 25 | SEQ ID NO: 50 |
| SEQ ID NO: 25 | SEQ ID NO: 51 |
| SEQ ID NO: 25 | SEQ ID NO: 52 |
| SEQ ID NO: 25 | SEQ ID NO: 53 |
| SEQ ID NO: 25 | SEQ ID NO: 58 |
| SEQ ID NO: 26 | SEQ ID NO: 31 |
| SEQ ID NO: 26 | SEQ ID NO: 32 |
| SEQ ID NO: 26 | SEQ ID NO: 33 |
| SEQ ID NO: 26 | SEQ ID NO: 34 |
| SEQ ID NO: 26 | SEQ ID NO: 35 |
| SEQ ID NO: 26 | SEQ ID NO: 36 |
| SEQ ID NO: 26 | SEQ ID NO: 37 |
| SEQ ID NO: 26 | SEQ ID NO: 38 |
| SEQ ID NO: 26 | SEQ ID NO: 39 |
| SEQ ID NO: 26 | SEQ ID NO: 40 |
| SEQ ID NO: 26 | SEQ ID NO: 41 |
| SEQ ID NO: 26 | SEQ ID NO: 42 |
| SEQ ID NO: 26 | SEQ ID NO: 43 |
| SEQ ID NO: 26 | SEQ ID NO: 44 |
| SEQ ID NO: 26 | SEQ ID NO: 45 |
| SEQ ID NO: 26 | SEQ ID NO: 46 |
| SEQ ID NO: 26 | SEQ ID NO: 47 |
| SEQ ID NO: 26 | SEQ ID NO: 48 |
| SEQ ID NO: 26 | SEQ ID NO: 49 |
| SEQ ID NO: 26 | SEQ ID NO: 50 |
| SEQ ID NO: 26 | SEQ ID NO: 51 |
| SEQ ID NO: 26 | SEQ ID NO: 52 |
| SEQ ID NO: 26 | SEQ ID NO: 53 |
| SEQ ID NO: 26 | SEQ ID NO: 58 |
| SEQ ID NO: 27 | SEQ ID NO: 31 |
| SEQ ID NO: 27 | SEQ ID NO: 32 |
| SEQ ID NO: 27 | SEQ ID NO: 33 |
| SEQ ID NO: 27 | SEQ ID NO: 34 |
| SEQ ID NO: 27 | SEQ ID NO: 35 |
| SEQ ID NO: 27 | SEQ ID NO: 36 |
| SEQ ID NO: 27 | SEQ ID NO: 37 |
| SEQ ID NO: 27 | SEQ ID NO: 38 |
| SEQ ID NO: 27 | SEQ ID NO: 39 |
| SEQ ID NO: 27 | SEQ ID NO: 40 |
| SEQ ID NO: 27 | SEQ ID NO: 41 |
| SEQ ID NO: 27 | SEQ ID NO: 42 |
| SEQ ID NO: 27 | SEQ ID NO: 43 |
| SEQ ID NO: 27 | SEQ ID NO: 44 |
| SEQ ID NO: 27 | SEQ ID NO: 45 |
| SEQ ID NO: 27 | SEQ ID NO: 46 |
| SEQ ID NO: 27 | SEQ ID NO: 47 |
| SEQ ID NO: 27 | SEQ ID NO: 48 |
| SEQ ID NO: 27 | SEQ ID NO: 49 |
| SEQ ID NO: 27 | SEQ ID NO: 50 |
| SEQ ID NO: 27 | SEQ ID NO: 51 |
| SEQ ID NO: 27 | SEQ ID NO: 52 |
| SEQ ID NO: 27 | SEQ ID NO: 53 |
| SEQ ID NO: 27 | SEQ ID NO: 58 |
| SEQ ID NO: 28 | SEQ ID NO: 31 |
| SEQ ID NO: 28 | SEQ ID NO: 32 |
| SEQ ID NO: 28 | SEQ ID NO: 33 |
| SEQ ID NO: 28 | SEQ ID NO: 34 |
| SEQ ID NO: 28 | SEQ ID NO: 35 |
| SEQ ID NO: 28 | SEQ ID NO: 36 |
| SEQ ID NO: 28 | SEQ ID NO: 37 |
| SEQ ID NO: 28 | SEQ ID NO: 38 |
| SEQ ID NO: 28 | SEQ ID NO: 39 |
| SEQ ID NO: 28 | SEQ ID NO: 40 |
| SEQ ID NO: 28 | SEQ ID NO: 41 |
| SEQ ID NO: 28 | SEQ ID NO: 42 |
| SEQ ID NO: 28 | SEQ ID NO: 43 |
| SEQ ID NO: 28 | SEQ ID NO: 44 |
| SEQ ID NO: 28 | SEQ ID NO: 45 |
| SEQ ID NO: 28 | SEQ ID NO: 46 |
| SEQ ID NO: 28 | SEQ ID NO: 47 |
| SEQ ID NO: 28 | SEQ ID NO: 48 |
| SEQ ID NO: 28 | SEQ ID NO: 49 |
| SEQ ID NO: 28 | SEQ ID NO: 50 |
| SEQ ID NO: 28 | SEQ ID NO: 51 |
| SEQ ID NO: 28 | SEQ ID NO: 52 |
| SEQ ID NO: 28 | SEQ ID NO: 53 |
| SEQ ID NO: 28 | SEQ ID NO: 58 |
| SEQ ID NO: 29 | SEQ ID NO: 31 |
| SEQ ID NO: 29 | SEQ ID NO: 32 |
| SEQ ID NO: 29 | SEQ ID NO: 33 |
| SEQ ID NO: 29 | SEQ ID NO: 34 |
| SEQ ID NO: 29 | SEQ ID NO: 35 |
| SEQ ID NO: 29 | SEQ ID NO: 36 |
| SEQ ID NO: 29 | SEQ ID NO: 37 |
| SEQ ID NO: 29 | SEQ ID NO: 38 |
| SEQ ID NO: 29 | SEQ ID NO: 39 |
| SEQ ID NO: 29 | SEQ ID NO: 40 |
| SEQ ID NO: 29 | SEQ ID NO: 41 |
| SEQ ID NO: 29 | SEQ ID NO: 42 |
| SEQ ID NO: 29 | SEQ ID NO: 43 |
| SEQ ID NO: 29 | SEQ ID NO: 44 |
| SEQ ID NO: 29 | SEQ ID NO: 45 |
| SEQ ID NO: 29 | SEQ ID NO: 46 |
| SEQ ID NO: 29 | SEQ ID NO: 47 |
| SEQ ID NO: 29 | SEQ ID NO: 48 |
| SEQ ID NO: 29 | SEQ ID NO: 49 |
| SEQ ID NO: 29 | SEQ ID NO: 50 |
| SEQ ID NO: 29 | SEQ ID NO: 51 |
| SEQ ID NO: 29 | SEQ ID NO: 52 |
| SEQ ID NO: 29 | SEQ ID NO: 53 |
| SEQ ID NO: 29 | SEQ ID NO: 58 |
| SEQ ID NO: 30 | SEQ ID NO: 31 |
| SEQ ID NO: 30 | SEQ ID NO: 32 |
| SEQ ID NO: 30 | SEQ ID NO: 33 |
| SEQ ID NO: 30 | SEQ ID NO: 34 |
| SEQ ID NO: 30 | SEQ ID NO: 35 |
| SEQ ID NO: 30 | SEQ ID NO: 36 |
| SEQ ID NO: 30 | SEQ ID NO: 37 |
| SEQ ID NO: 30 | SEQ ID NO: 38 |
| SEQ ID NO: 30 | SEQ ID NO: 39 |
| SEQ ID NO: 30 | SEQ ID NO: 40 |
| SEQ ID NO: 30 | SEQ ID NO: 41 |
| SEQ ID NO: 30 | SEQ ID NO: 42 |
| SEQ ID NO: 30 | SEQ ID NO: 43 |
| SEQ ID NO: 30 | SEQ ID NO: 44 |
| SEQ ID NO: 30 | SEQ ID NO: 45 |
| SEQ ID NO: 30 | SEQ ID NO: 46 |
| SEQ ID NO: 30 | SEQ ID NO: 47 |
| SEQ ID NO: 30 | SEQ ID NO: 48 |
| SEQ ID NO: 30 | SEQ ID NO: 49 |
| SEQ ID NO: 30 | SEQ ID NO: 50 |
| SEQ ID NO: 30 | SEQ ID NO: 51 |
| SEQ ID NO: 30 | SEQ ID NO: 52 |
| SEQ ID NO: 30 | SEQ ID NO: 53 |
| SEQ ID NO: 30 | SEQ ID NO: 58 |
| SEQ ID NO: 56 | SEQ ID NO: 31 |
| SEQ ID NO: 56 | SEQ ID NO: 32 |
| SEQ ID NO: 56 | SEQ ID NO: 33 |
| SEQ ID NO: 56 | SEQ ID NO: 34 |
| SEQ ID NO: 56 | SEQ ID NO: 35 |
| SEQ ID NO: 56 | SEQ ID NO: 36 |
| SEQ ID NO: 56 | SEQ ID NO: 37 |
| SEQ ID NO: 56 | SEQ ID NO: 38 |
| SEQ ID NO: 56 | SEQ ID NO: 39 |
| SEQ ID NO: 56 | SEQ ID NO: 40 |
| SEQ ID NO: 56 | SEQ ID NO: 41 |
| SEQ ID NO: 56 | SEQ ID NO: 42 |
| SEQ ID NO: 56 | SEQ ID NO: 43 |
| SEQ ID NO: 56 | SEQ ID NO: 44 |
| SEQ ID NO: 56 | SEQ ID NO: 45 |
| SEQ ID NO: 56 | SEQ ID NO: 46 |
| SEQ ID NO: 56 | SEQ ID NO: 47 |
| SEQ ID NO: 56 | SEQ ID NO: 48 |
| SEQ ID NO: 56 | SEQ ID NO: 49 |
| SEQ ID NO: 56 | SEQ ID NO: 50 |
| SEQ ID NO: 56 | SEQ ID NO: 51 |
| SEQ ID NO: 56 | SEQ ID NO: 52 |
| SEQ ID NO: 56 | SEQ ID NO: 53 |
| SEQ ID NO: 56 | SEQ ID NO: 58 |
| SEQ ID NO: 57 | SEQ ID NO: 31 |
| SEQ ID NO: 57 | SEQ ID NO: 32 |
| SEQ ID NO: 57 | SEQ ID NO: 33 |
| SEQ ID NO: 57 | SEQ ID NO: 34 |
| SEQ ID NO: 57 | SEQ ID NO: 35 |
| SEQ ID NO: 57 | SEQ ID NO: 36 |
| SEQ ID NO: 57 | SEQ ID NO: 37 |
| SEQ ID NO: 57 | SEQ ID NO: 38 |
| SEQ ID NO: 57 | SEQ ID NO: 39 |
| SEQ ID NO: 57 | SEQ ID NO: 40 |
| SEQ ID NO: 57 | SEQ ID NO: 41 |
| SEQ ID NO: 57 | SEQ ID NO: 42 |
| SEQ ID NO: 57 | SEQ ID NO: 43 |
| SEQ ID NO: 57 | SEQ ID NO: 44 |
| SEQ ID NO: 57 | SEQ ID NO: 45 |
| SEQ ID NO: 57 | SEQ ID NO: 46 |
| SEQ ID NO: 57 | SEQ ID NO: 47 |
| SEQ ID NO: 57 | SEQ ID NO: 48 |
| SEQ ID NO: 57 | SEQ ID NO: 49 |
| SEQ ID NO: 57 | SEQ ID NO: 50 |
| SEQ ID NO: 57 | SEQ ID NO: 51 |
| SEQ ID NO: 57 | SEQ ID NO: 52 |
| SEQ ID NO: 57 | SEQ ID NO: 53 |
| SEQ ID NO: 57 | SEQ ID NO: 58 |

In a preferred embodiment, the recombinant construct comprises a ribosome binding site comprising SEQ ID NO: 24 operably linked to a synthetic front end of a prolactin gene comprising a sequence selected from the group consisting of SEQ ID NOs: 31-36. In another preferred embodiment, the recombinant construct comprises a ribosome binding site comprising SEQ ID NO: 25 operably linked to a synthetic front end of a prolactin gene comprising SEQ ID NO: 37. In another preferred embodiment, the recombinant construct comprises a ribosome binding site comprising SEQ ID NO: 26 operably linked to a synthetic front end of a prolactin gene comprising a sequence selected from the group consisting of SEQ ID NOs: 38-46. In another preferred embodiment, the recombinant constructs comprises a ribosome binding site comprising SEQ ID NO: 27 operably linked to a synthetic front end of a prolactin gene comprising SEQ ID NO: 47. In another preferred embodiment, the recombinant construct comprises a ribosome binding site comprising SEQ ID NO: 28 operably linked to a synthetic front end of a prolactin gene comprising SEQ ID NO: 48. In another preferred embodiment, the recombinant construct comprises a ribosome binding site comprising SEQ ID NO: 29 operably linked to a synthetic front end of a prolactin gene comprising a sequence selected from the group consisting of SEQ ID NOs: 49-52. In another embodiment, the recombinant construct comprises a ribosome binding site comprising SEQ ID NO: 30 operably linked to a synthetic front end of a prolactin gene comprising SEQ ID NO: 53.

Another aspect of the invention is a transformed host cell comprising a recombinant construct including a ribosome binding site selected from SEQ ID NOs: 24-30, 56, and 57 operably linked to a promoter. In certain embodiments, the transformed host cell comprises a recombinant construct comprising a ribosome binding site operably linked to a synthetic front end of a prolactin gene and a promoter. In particular embodiments, the transformed host cell comprises any one of the recombinant constructs disclosed herein, and preferably a construct disclosed in Table 1. In some embodiments, the host cell is a prokaryotic cell. In preferred embodiments, the host cell is an Escherichia coli cell.

Another aspect of the invention is a method for producing a prolactin antagonist in a transformed host cell, and in particular in a transformed host cell containing a recombinant construct disclosed herein. These methods include culturing a host cell comprising a ribosome binding site selected from the group consisting of SEQ ID NOs: 24-30, 56, and 57 operably linked to a synthetic front end of a prolactin gene under conditions that induce gene expression from the prolactin antagonist DNA sequence. In a preferred embodiment, the host cell comprises a ribosome binding site selected from the group consisting of SEQ ID NOs: 24-30, 56 and 57 operably linked to a synthetic front end of a prolactin gene selected from the group consisting of SEQ ID NOs: 31-53 and 58. In another embodiment, the host cell comprises a ribosome binding site operably linked to a synthetic front end of a prolactin gene according to any one of the combinations listed in Table 1. In preferred embodiments, the prolactin antagonist produced is the G129R variant human prolactin antagonist as disclosed, for example, in U.S. Pat. No. 7,115,556.

Another aspect of the invention is isolated nucleic acid sequences, and in particular, plasmids comprising the sequences, which comprise a sequence selected from the group consisting of SEQ ID NOs: 1-23, 54, and 55.

Another aspect of the invention is recombinant constructs comprising a ribosome binding site sequence operably linked to a prolactin antagonist DNA sequence, wherein the construct comprises a sequence selected from the group consisting of SEQ ID NOs: 1-23, 54, and 55. In some embodiments, the recombinant construct is operably linked to a promoter functional in a host cell. In preferred embodiments, the prolactin antagonist DNA sequence encodes the G129R variant human prolactin antagonist as disclosed, for example, in U.S. Pat. No. 7,115,556.

Another aspect of the invention is a transformed host cell which comprises a recombinant construct comprising a ribosome binding site sequence operably linked to a prolactin antagonist DNA sequence, wherein the construct comprises a sequence selected from the group consisting of SEQ ID NOs: 1-23, 54, and 55. In some embodiments, the recombinant construct is operably linked to a promoter functional in a host cell. In some embodiments, the host cell is a prokaryotic cell. In preferred embodiments, the host cell is an Escherichia coli cell. In preferred embodiments, the prolactin antagonist DNA sequence encodes the G129R variant human prolactin antagonist as disclosed, for example, in U.S. Pat. No. 7,115,556.

Another aspect the invention is methods for producing a prolactin antagonist in a transformed host cell, wherein the methods include culturing a host cell comprising a nucleic acid sequence selected from the group consisting of SEQ ID NOs: 1-23, 54, and 55 under conditions that induce gene expression from the prolactin antagonist DNA sequence. In preferred embodiments of this method, the prolactin antagonist is the G129R variant human prolactin antagonist.

Each of the vectors having SEQ ID NOs: 1-23 includes a synthetic promoter, designated “cpex-20”, which is disclosed in U.S. Pat. No. 6,617,130, the entire content of which is hereby incorporated herein by reference. This promoter is situated between the EcoRI site (GAATTC) at the beginning of each sequence and the AscI site (GGCGCGCC) at coordinate 76 of each sequence. Any number of well known bacterial promoters (e.g., Lisser and Margalit, 1993; Chasov et al, 2002; and U.S. Patent Publication No. US 2009/0093023), including any of a number of various other conventional and novel promoters, can be used in these vectors in lieu of the cpex-20 promoter to achieve good levels of expression of human prolactin antagonists. For example, each of the vectors having SEQ ID NOs 54-55 includes the rightward promoter from bacteriophage lambda, designated the PR promoter, situated before the ribosome binding site and prolactin gene.

Other promoters could be used, such as for example promoters from chromosomal genes of Escherichia coli, such as the tip promoter, the lac promoter, the recA promoter, the ara promoter, the gal promoter, the phoA promoter, and the lpp promoter, and derivatives of these promoters such as the lacUV5 promoter and the tac promoter, as well as promoters derived from bacteriophage genomes, such as the bacteriophage lambda leftward promoter (the PL promoter), and equivalents thereof. Another promoter that could be used is the bacteriophage T7 promoter that is employed on the pET family of plasmid vectors sold by Novagen and disclosed in U.S. Pat. No. 4,952,496, herein incorporated by reference. One advantage of the cpex-20 promoter, as disclosed in U.S. Pat. No. 6,617,130, is that it is a regulated promoter which can be induced (i.e., “turned on”) by the inexpensive, safe, and readily available chemical inducer nalidixic acid.

Each of the vectors having SEQ ID NOs: 1-23 also includes a synthetic transcription terminator, designated “double lac”, which is disclosed in U.S. Pat. No. 6,828,124, the entire content of which is hereby incorporated herein by reference. This transcription terminator is situated between the XhoI site (CTCGAG), located at approximately coordinate 715 of the sequences, and the HindIII site (AAGCTT) located at the end of the sequences. Various other well known conventional and novel transcription terminators can be used in these vectors in lieu of the double lac transcription terminator to achieve good levels of expression of human prolactin antagonists. For example, each of the vectors having SEQ ID NOs: 54-55 includes a synthetic transcription terminator, designated “pot3”.

Other transcription terminators could be used, such as for example transcription terminators from chromosomal genes of Escherichia coli, such as the rrnB, the trp, the gal, and the lac transcription terminators, as well as transcription terminators derived from bacteriophage genomes, such as the bacteriophage fd transcription terminator, and bacteriophage lambda transcription terminators, and equivalents thereof. While the inclusion of a transcription terminator was found to enhance the expression of human prolactin antagonists, a transcription terminator is not required for high level expression of the protein. Thus, it would also be possible to omit the inclusion of a transcription terminator in these vectors in lieu of the double lac transcription terminator.

Structural genes encoding the G129R variant human prolactin antagonist, preceded by the cpex-20 promoter and a ribosome binding site, and followed by the double lac transcription terminator, were prepared by DNA synthesis as EcoRI-HindIII restriction fragments (SEQ ID NOs: 1 through 23). Structural genes encoding the G129R variant human prolactin antagonist, preceded by the PR promoter and a ribosome binding site, and followed by the pot3 transcription terminator, were prepared by DNA synthesis as ClaI-SalI restriction fragments (SEQ ID NOs: 54 and 55). This technique of DNA synthesis is known in the art. See, for example, Khudyakov and Fields, 2003, the entire content of which is hereby incorporated herein by reference.

The synthetic EcoRI-HindIII restriction fragments, carrying the cpex-20 promoter, a ribosome binding site, the entire human prolactin antagonist structural gene, and a transcription terminator, were inserted between the EcoRI and HindIII restriction sites on the standard cloning vector pBR322 (Bolivar et al, 1977; Pouwels et al., 1985; Balbas et al., 1986; Balbas et al., 1988) to yield the vectors listed in Table 2 with SEQ ID NOs: 1-23. The synthetic ClaI-SalI restriction fragments, carrying the PR promoter, a ribosome binding site, the entire human prolactin antagonist structural gene, and a transcription terminator, were inserted between the ClaI and SalI restriction sites on the standard cloning vector pBR327 (Bolivar et al, 1977; Pouwels et al., 1985; Balbas et al., 1986; Balbas et al., 1988) to yield the vectors listed in Table 2 with SEQ ID NOs: 54-55. The techniques of manipulation of DNA molecules, including the cleavage of DNA molecules with restriction enzymes and the ligation of restriction fragments of DNA, are known in the art. See, for example, Sambrook and Russell, 2001, and Ausubel et al., 2005, each of which are hereby incorporated herein by reference in their entirety. Other plasmids are known and regularly used by ordinarily skilled artisans as cloning vectors suitable for transforming Escherichia coli and/or other bacteria, and which can also be used to achieve expression of human prolactin antagonists in the transformed bacteria. Detailed guides to such cloning vectors are known in the art; see for example, Pouwels et al., 1985, and Balbas et al., 1988, each of which are hereby incorporated herein by reference in their entirety. Techniques for transforming bacteria, including strains of Escherichia coli, are also well known in the art; see for example Sambrook and Russell, 2001, and Ausubel et al., 2005, each of which are hereby incorporated herein by reference.

TABLE 2
Exemplary list of vectors

| SEQ ID NO | RBS | Plasmid Name |
|---|---|---|
| 1 | gene 10a | pXT1520 |
| 2 | gene 10a | pXT1521 |
| 3 | gene 10a | pXT1522 |
| 4 | gene 10a | pXT1523 |
| 5 | gene 10a | pXT1526 |
| 6 | gene 10a | pXT1528 |
| 7 | hpr-02 | pXT1577 |
| 8 | hpr-03 | pXT1578 |
| 9 | hpr-03 | pXT1578 |
| 10 | hpr-03 | pXT1580 |
| 11 | hpr-03 | pXT1581 |
| 12 | hpr-03 | pXT1582 |
| 13 | hpr-03 | pXT1583 |
| 14 | hpr-03 | pXT1584 |
| 15 | hpr-03 | pXT1585 |
| 16 | hpr-03 | pXT1588 |
| 17 | hpr-06 | pXT1572 |
| 18 | hpr-13 | pXT1576 |
| 19 | hpr-17 | pXT1586 |
| 20 | hpr-17 | pXT1589 |
| 21 | hpr-17 | pXT1590 |
| 22 | hpr-17 | pXT1591 |
| 23 | hpr-18 | pXT1587 |
| 54 | gene 10a | pXT1600 |
| 55 | hpr-17 | pXT1614 |

SEQ ID NOs: 1 through 6 plus SEQ ID NO: 54 share a novel synthetic ribosome binding site, TTAACTTTAAGAAGGAGGAAAAAAT (SEQ ID NO: 24), herein designated gene 10a.

SEQ ID NO: 7 has a second novel synthetic ribosome binding site, GATGAAATAGGAGGAACAACA (SEQ ID NO: 25), herein designated hpr-02.

SEQ ID NOs: 8 through 16 share a third novel synthetic ribosome binding site, AATGAAATAGGAGGATAATTT (SEQ ID NO: 26), herein designated hpr-03.

SEQ ID NO: 17 has a fourth novel synthetic ribosome binding site, CTTAATTAAGGAGGTAAATTA (SEQ ID NO: 27), herein designated hpr-06.

SEQ ID NO: 18 has a fifth novel synthetic ribosome binding site, GAATTTAATGGAGGAAGAAAAGA (SEQ ID NO: 28), herein designated hpr-13.

SEQ ID NOs: 19-22 plus SEQ ID NO: 55 share a sixth novel synthetic ribosome binding site, AAATTAATAGGAGGAATTTAGAT (SEQ ID NO: 29), herein designated hpr-17.

SEQ ID NO: 23 has a seventh novel synthetic ribosome binding site, GAATATAAAGGAGGAATTTTATT (SEQ ID NO: 30), herein designated hpr-18.

The novel synthetic ribosome binding sequences listed above, while each having its own specific sequence, can generally be represented by the consensus sequence VWWDWWWWWGGAGGWWWWWWW (SEQ ID NO: 56) or VWWDWWWWWGGAGGWWWWWWWWW (SEQ ID NO: 57), wherein V, W, and D represent nucleotides in accordance with the IUPAC codes for nucleotide sequences. That is to say, a nucleotide at a position represented by V may be an adenine (A), a guanine, (G), or a cytosine (C); a nucleotide at a position represented by W may be an adenine (A) or a thymine (T); and a nucleotide at a position represented by D may be an adenine (A), a thymine (T), or a guanine (G). Accordingly, another aspect of the invention is an isolated synthetic ribosome binding sequence. In one embodiment, the synthetic ribosome binding sequence comprises a nucleotide sequence comprising the sequence VWWDWWWWWGGAGGWWWWWWW (SEQ ID NO: 56). In another embodiment, the synthetic ribosome binding sequence comprises a nucleotide sequence comprising the sequence VWWDWWWWWGGAGGWWWWWWWWW (SEQ ID NO: 57). In yet another embodiment, the synthetic ribosome binding sequence is VWWDWWWWWGGAGGWWWWWWW (SEQ ID NO: 56). In yet another embodiment, the synthetic ribosome binding sequence is VWWDWWWWWGGAGGWWWWWWWWW (SEQ ID NO: 57). In another embodiment, the isolated synthetic ribosome binding sequence comprises a nucleotide sequence selected from the group consisting of SEQ ID NOS: 24, 25, 26, 27, 28, 29, and 30. In another embodiment, the synthetic ribosome binding sequence is a nucleotide sequence selected from the group consisting of SEQ ID NOS: 24, 25, 26, 27, 28, 29, and 30. Each of these isolated synthetic ribosome binding sequences may be operably linked to a synthetic front end of a prolactin gene, such as those represented by SEQ ID NOs: 31-53, to form an isolated sequence comprising a ribosome binding site and a synthetic front end of a prolactin gene useful for creating recombinant constructs for producing prolactin and/or prolactin antagonists.

Another aspect of the present invention is a nucleic acid sequence comprising a synthetic ribosome binding site and a synthetic front end of a prolactin gene. Combinations of the synthetic ribosome binding site and the synthetic front end of a prolactin gene may be any one of those as disclosed herein, and in particular, any one of those disclosed in Table 1. In a particular embodiment, the synthetic ribosome binding site is operably linked to the synthetic front end of a prolactin gene.

In each of SEQ ID NOs: 1 through 23, each ribosome binding site is situated between the AscI site (GGCGCGCC) and the ATG translation start codon of the human prolactin antagonist structural gene. The AscI site begins at coordinate 76 of each sequence. The ATG translational start codon begins at approximately coordinate 105; this location varies slightly due the ribosome binding sites being employed having slightly different lengths. In each of SEQ ID NOs: 54 through 55, each ribosome binding site is situated upstream of the ATG translation start codon of the human prolactin antagonist structural gene. The ATG translational start codon begins at coordinate 900 of SEQ ID NO: 54, and at coordinate 898 of SEQ ID NO: 55.

Embodiments of the present invention, including all of the vectors listed in Table 2, have structural genes that differ from cDNA encoding human prolactin. First, they all contain a change in codon 129 from glycine GGC to arginine CGC. This alteration changes the wild-type human prolactin protein into the G129R variant human prolactin antagonist. Second, they all contain various arrays of silent changes within the nucleotides encoding the first 11 codons of the human prolactin structural gene. This altered region is termed the “synthetic front end” of the human prolactin antagonist structural gene. These synthetic front ends of the human prolactin antagonist structural gene, coupled with the novel synthetic ribosome binding sites disclosed herein, were found to lead to high-level expression of human prolactin antagonist protein. The level of the human prolactin antagonist protein expression was measured using an HPLC assay. This HPLC assay is described in Bogosian et al. 1989, where it was used to measure growth hormone expression in Escherichia coli cells, and which is herein incorporated by reference.

Another aspect of the present invention comprises synthetic front ends of the prolactin gene. These synthetic front ends are described in SEQ ID NOs: 31-53 and 58. In a particular embodiment, the synthetic front end of the prolactin gene is represented by the consensus sequence ATGYTRCCRATTTGYCCRGGYGGDGCHGCVMGRTGY (SEQ ID NO: 58) wherein Y, R, D, H, V, and M represent nucleotides in accordance with the IUPAC codes for nucleotide sequences. That is to say, a nucleotide at a position represented by Y may be a cytosine (C) or a thymine (T); a nucleotide at a position represented by R may be an adenine (A) or a guanine (G); a nucleotide at a position represented by D may be an adenine (A), a thymine (T), or a guanine (G); a nucleotide at a position represented by H may be an adenine (A), a thymine (T), or a cytosine (C); a nucleotide at a position represented by V may be an adenine (A), a guanine, (G), or a cytosine (C); and a nucleotide at a position represented by M may be an adenine (A) or a cytosine (C). Any one of the synthetic front ends of a prolactin gene as disclosed herein may be operably linked with a synthetic ribosome binding site disclosed here, further operably linked with a promoter, and further operably linked with a nucleotide sequence encoding prolactin or a prolactin antagonist. While not wishing to be bound by theory, it is believed that these synthetic front ends of the prolactin gene, coupled with the novel synthetic ribosome binding sites disclosed herein, are important elements of the function of the expression vectors of embodiments of the present invention.

In another aspect, the invention comprises methods of preparing, recovering, and purifying human prolactin antagonists, and particularly the G129R variant human prolactin antagonist, produced by recombinant expression systems such as those more particularly described herein, and in particular, through the use of the recombinant constructs, vectors, and plasmids described herein, such as, for example, in Tables 1 and 2. When recombinant proteins are expressed from transformed host cells as described herein, the desired expressed protein is present in the fermentation medium as inclusion bodies (i.e., cytoplasmic aggregates and oligomers containing the human prolactin antagonist protein to be recovered) in the host cell culture. Applicants have found that human prolactin antagonists can be recovered and purified from the expression systems of the present invention by methods generally comprising isolating the inclusion bodies from the host cell culture, refolding the isolated inclusion bodies, filtering the refold, acid precipitating impurities, and recovering the purified proteins by anion exchange chromatography. Theses methods are similar to those as described in U.S. Patent Publication No. US 2009/0093023, the entire content of which is hereby incorporated herein by reference. Applicants have discovered that the recovery and purification processes of the present invention provide for a simple robust process, high product purity, and low production cost.

The first step of the recovery and purification process after fermentation comprises isolating inclusion bodies from the fermentation contents. Typically, the inclusion bodies are purified by high pressure homogenization and differential centrifugation. In a particular embodiment, the fermenter contents are contacted with tetrabasic EDTA after fermentation. The fermentation broth is then cooled to less than 25° C., typically 5-10° C., and either homogenized directly or centrifuged to collect the whole cells. The isolation process may be continued over several iterations to further isolate and purify the inclusion bodies by washing the inclusion body slurry with cold de-ionized water and repeating the homogenization and centrifugation steps. The final, purified inclusion body slurry can be held cold for several days or frozen at −80° C. for longer term storage.

After isolation, the inclusion bodies are solubilized and the human prolactin antagonist proteins are refolded. As used herein “refolding” includes the steps of “protein folding” or the return of the overall conformational shape of the protein and “oxidation” which is the formation of the intramolecular disulfide bonds generally required for a biologically active conformation. Upon refolding, the protein is preferably in its native biologically active conformation.

In a particular embodiment for the human prolactin antagonist proteins described herein, the isolated inclusion bodies may be dissolved cold (at a temperature of from about 5° C. to about 10° C.) at a concentration of about 10 g/L in a solution of 3% acylglutamate (Ajinomoto) adjusted to a final pH of 11.0 with a base such as sodium hydroxide. After dissolution of the inclusion bodies, the protein is oxidized. Approximately 15-30 minutes after dissolution the solution is diluted with an equal volume of cold water. Cystine solids are added to a final concentration of about 1 mM and the pH is readjusted to 11.0 with sodium hydroxide. The resulting solution is kept cold (at a temperature of from about 5° C. to about 10° C.) and mixed for 18 hours. At lab scale the beaker is covered to prevent uptake of carbon dioxide thereby avoiding a drop in pH. At large scale, the solution is slowly purged with air to replace oxygen used in the reaction.

After refolding, the finished refold solution is concentrated and filtered to begin impurity precipitation. Preferably, he finished refold solution is concentrated and diafiltered versus 10 turnover volumes of cold 1 mM NaOH while keeping the solution at a temperature of from about 5° C. to about 10° C. as described above to remove any detergent which may interfere with the impurity precipitation. A suitable membrane for the diafiltration is a 10,000 MWt cutoff regenerated cellulose.

After diafiltration, the recovered refold is precipitated with acid. In a preferred embodiment, the diafiltered refold is diluted to about 20 g/L total protein with water. With good mixing, 5% acetic acid is added over 30 minutes to a final pH of 4.5. The suspension is mixed for an additional 5-10 minutes before clarification.

At lab scale the precipitate suspension is centrifuged to clarify. At production scale the suspension is flocculated by contacting the suspension with a polymer solution. The flocculated solution is gravity settled and the clear supernatant is decanted. All operations are cold with the exception of the polymer solution which is prepared and used at room temperature.

In a preferred embodiment, a 150 ppm stock solution of polymer (Chemtall Floerger AN905 polymer) is prepared by adding the polymer to water with very rapid mixing. The polymer solution is added to the acid precipitated suspension slowly over 30 minutes with gentle mixing to a final concentration of 50 ppm. Mixing is continued for 5 minutes and then stopped to allow the suspension to gravity settle. After settling, the clear upper supernatant is decanted.

To further recover soluble protein trapped in the settled solids, it may be desirable or preferred to further wash the settled solids. The solids may be washed by contacting with a sufficient volume water to restore the original flocculation volume. The re-suspended solids may be then further contacted with polymer (approximately 2 ppm final concentration), settled, and the clear supernatant is decanted. For additional yield recovery a second wash is preferably performed. In an alternative embodiment, the acid precipitation step can be skipped and diafiltered refold solution can be loaded directly onto the chromatography column.

After precipitation, the prolactin antagonist proteins are separated and recovered from the decanted supernatant by chromatography. The decanted acid precipitation supernatant is cooled to a temperature of from about 5° C. to about 10° C. and the pH is adjusted to about 10 with NaOH. The cooled supernatant is concentrated by ultrafiltration membrane and diafiltered versus 3-5 turnover volumes of cold 1 mM NaOH. A suitable membrane for diafiltration is a 10,000 MWt cutoff polyether sulfone membrane works.

The pH adjusted clarified acid precipitation supernatant is loaded to a chromatography column to 25 g/L total protein and the column is washed with a buffer solution. Preferably, the chromatography buffer used is 4.5 M urea, 50 mM Tris base. The urea is ultrapure grade and then further purified by mixed bed deionization prior to use. Care is taken at small scale to keep the Tris base from exposure to excess CO2 from the air which will lower its pH. Urea solutions and urea buffers are stored cold. Suitable chromatography resins for the column include Whatman DE-52 cellulose and Pall Biosepra DEAE-Spherodex. The resin should be equilibrated before loading with 0.1M NaOH in 4.5 M urea followed by 3-5 column volumes of 4.5 M urea, 50 mM Tris base (to a conductivity of <100 uS/cm). The loaded column is then eluted with a linear gradient from 4.5 M urea, 50 mM Tris base to 4.5 M urea, 50 mM TrisCl over 20 column volumes. Fractions are collected, analyzed and cut according to the desired product purity.

Having described the invention in detail, it will be apparent that modifications and variations are possible without departing the scope of the invention defined in the appended claims. Furthermore, it should be appreciated that all examples in the present disclosure are provided as non-limiting examples.

### EXAMPLES

The following non-limiting examples are provided to further illustrate the present invention. It should be appreciated by those of skill in the art that the techniques disclosed in the examples that follow represent approaches the inventors have found function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Expression of Variant Human Prolactin Antagonist Protein in Escherichia coli Under Control of the cpex-20 Promoter

The standard wild-type Escherichia coli K-12 host strain W3110 was used in experiments to determine the expression level of the G129R variant human prolactin antagonist protein. The strain W3110 was transformed with the plasmid pXT 1520 listed in Table 2 (SEQ ID NO: 1). The strain W3110 carrying the plasmid pXT1520 was grown in a fermentation vessel and induced for expression of the G129R variant human prolactin antagonist protein.

The fermentation was conducted in a chemically-defined minimal medium containing 5.6 grams of anhydrous ammonium sulfate ((NH₄)₂SO₄), 6.7 grams of anhydrous dibasic potassium phosphate (K₂HPO₄), 3.3 grams of monobasic sodium phosphate monohydrate (NaH₂PO₄—H₂O), 530 milligrams of monobasic magnesium phosphate (Mg(H₂PO₄)₂), 6 milligrams of ferric chloride hexahydrate (FeCl₃—6H₂O), 0.4 milligrams of zinc sulfate heptahydrate (ZnSO₄—7H₂O), 0.8 milligrams of cobalt chloride hexahydrate (CoCl₂—6H₂O), 0.8 milligrams of sodium molybdate dihydrate (Na₂MoO₄—2H₂O), 0.9 milligrams of cupric sulfate pentahydrate (CuSO₄—5H₂O), 0.2 milligrams of boric acid (H₃BO₃), and 0.6 milligrams of manganese sulfate monohydrate (MnSO₄—H₂O) per liter of water. There were no antibiotics added to the fermentation medium. The fermenter was maintained at 37 degrees Celsius. The pH was maintained at 7.0 by the controlled addition of concentrated (about 29%) ammonium hydroxide (NH₄OH). Glucose was fed at a controlled rate from a 50% stock solution to maintain a glucose concentration of 2 grams per liter. When the culture reached an optical density at 660 nm of 45, synthesis of the G129R variant human prolactin antagonist protein was induced by the addition of nalidixic acid to a final concentration of 50 milligrams per liter. The induced fermentation culture was maintained for an additional 8 hours before being harvested. Analysis of a sample of the culture by an HPLC assay indicated that the G129R variant human prolactin antagonist protein was expressed at a level of about 2.2 grams per liter.

Each of the vectors comprising SEQ ID Nos 1-23 were transformed into Escherichia coli and found to express the G129R variant human prolactin antagonist protein at unoptimized levels of at least about 1.5 grams per liter, and most of them above 2.0 grams per liter, using similar methods.

### Example 2

### Expression of Variant Human Prolactin Antagonist Protein in Escherichia coli Under Control of the PR Promoter

The standard wild-type Escherichia coli K-12 host strain W3110 was used in experiments to determine the expression level of the G129R variant human prolactin antagonist protein. The strain W3110 was transformed with the plasmid pXT 1600 listed in Table 2 (SEQ ID NO: 54). The strain W3110 carrying the plasmid pXT1600 was grown in a fermentation vessel and induced for expression of the G129R variant human prolactin antagonist protein.

The fermentation was conducted in a chemically-defined minimal medium containing 5.6 grams of anhydrous ammonium sulfate ((NH₄)₂SO₄), 6.7 grams of anhydrous dibasic potassium phosphate (K₂HPO₄), 3.3 grams of monobasic sodium phosphate monohydrate (NaH₂PO₄—H₂O), 530 milligrams of monobasic magnesium phosphate (Mg(H₂PO₄)₂), 6 milligrams of ferric chloride hexahydrate (FeCl₃—6H₂O), 0.4 milligrams of zinc sulfate heptahydrate (ZnSO₄—7H₂O), 0.8 milligrams of cobalt chloride hexahydrate (CoCl₂—6H₂O), 0.8 milligrams of sodium molybdate dihydrate (Na₂MoO₄—2H₂O), 0.9 milligrams of cupric sulfate pentahydrate (CuSO₄—5H₂O), 0.2 milligrams of boric acid (H₃BO₃), and 0.6 milligrams of manganese sulfate monohydrate (MnSO₄—H₂O) per liter of water. There were no antibiotics added to the fermentation medium. The fermenter was maintained at 35 degrees Celsius. The pH was maintained at 7.0 by the controlled addition of concentrated (about 29%) ammonium hydroxide (NH₄OH). Glucose was fed at a controlled rate from a 50% stock solution to maintain a glucose concentration of 2 grams per liter. When the culture reached an optical density at 660 nm of 30, synthesis of the G129R variant human prolactin antagonist protein was induced by increasing the temperature of the fermentation culture to 42 degrees Celsius for one hour, and then reducing the temperature of the fermentation culture to 40 degrees Celsius. The induced fermentation culture was maintained for an additional 7 hours before being harvested. Analysis of a sample of the culture by an HPLC assay indicated that the G129R variant human prolactin antagonist protein was expressed at a level of about 4.0 grams per liter.

Each of the vectors comprising SEQ ID Nos 54 through 55 were transformed into Escherichia coli and found to express the G129R variant human prolactin antagonist protein at unoptimized levels of at least about 4.0 grams per liter.

### Example 3

### Inclusion Body Isolation Using Difference Centrifugation

The contents of a 10 L of prolactin fermentation were high pressure homogenized with two passes at 25,000 psi with a Microfluidics microfluider. The second pass broth homogenate was transferred into 1-liter centrifuge bottles and centrifuged in JLA-8.1000 rotor at 7000 rpm and 4° C. for 15 minutes. Supernatant was poured off and discarded. Pellets were re-suspended in cold water (−350 ml) using a Tekmar Turrax mixer. The re-suspended contents were passed through the microfluidizer again at 25,000 psi for a single pass. The wash homogenate was centrifuged JLA-8.1000 rotor at 7000 rpm and 4° C. for 15 minutes. After centrifuge, the pellet was re-suspended and washed once more, then final re-suspension was done by adding 200 ml of cold water to the pellets with Turrax at low speed. The re-suspended inclusion body slurry was frozen at −80° C. for storage.

### Example 4

### Inclusion Body Dissolution and Protein Refolding

Prolactin inclusion bodies (52 ml, 96 mg/ml) were added to a solution containing water (388 ml) and acylglutamate (59 ml of Ajinomoto 22% LS-22). This mixture was mixed for 30 minutes without pH adjustment (pH 7.5) and at a temp of 8° C. After 30 min, 5.8 ml of 1N NaOH were added slowly to adjust the pH to 11.0. After 30 min, 500 ml of cold deionized water were added followed by 6.7 ml cystine stock solution (150 mM adjusted to pH 10.5 with NaOH). The final prolactin concentration was 5 mg/ml and the final cystine concentration was 1 mM. The pH was maintained at 11.0 and mixed overnight for completion.

Alternatively, the inclusion bodies can be solubilized in 3-4.5M urea at pH 11 and mixed to refold and oxidize.

### Example 5

### Refold Ultrafiltration

Finished refold was filtered through 0.22 μM filters before diafiltration using a Millipore 6 ft² 10 kD MWCO regenerated cellulose cartridge (Prep/Scale-TFF, PN SK1P003W4, Lot no. C7DN47807). The operation was conducted at 4.5-6° C. with ice water bath. The initial filtered refold (981 gm) was concentrated to approximately 850 ml before diafiltration against 1 mM NaOH for 16 TOV.

### Example 6

### Acid Precipitation

Acid precipitation was conducted at 2 mg/ml at room temperature at a 200 ml volume. Dilute acetic acid (1%) was used to lower the pH to an endpoint of pH 4.5. The precipitated solution was centrifuged (2700K rpm/9° C. for 10 min) and the supernatant was filtered through a 0.45μ filter. This solution (180 ml) was dialyzed using 10 kD MWCO membrane (Pierce snake skin bag) with three buffer exchanges (14 liters each) against 0.1 mM NaOH. (NBP7947618)

### Example 7

### Anion Exchange Chromatography

Anion exchange chromatography was performed using a BioRad column (1.0 cm id×30 cm ht, PN 737-1031) packed with DE-52 resin. The column was packed in water (1.5 ml/min) and 0.1N NaOH (0.29 ml/min) with a flow adaptor (PN 738-0015) and a funnel (PN 731-0001) to ensure packing homogeneity. After NaOH packing, the column was equilibrated with Buffer A (0.29 ml/min) The final column height was 21 cm with a column volume (CV) of 16.5 ml.

All chromatography was performed using an AKTA Explorer 100 with a flow rate of 0.29 ml/min (1.05 CV/hr) at 4-5° C. Load sample was prepared by adding 270 ml of de-ionized 7.5M urea to the dialyzed acid precipitation pool (180 ml, 1.24 mg/ml) for a 4.5M urea load sample. The total load of protein was 208 mg in 420 ml. The column was first equilibrated with 3 CV of Buffer A before loading. At the end of loading, column was washed with 1 CV of Buffer A before the start of a linear gradient elution. The elution was performed with a 15 CV gradient from Buffer A to Buffer B. At the end of the gradient, an additional 5 CV of Buffer B was applied. Column elution was monitored by A₂₈₀ and the eluent was collected using a fraction collector (Frac-950) and a 3 ml per tube collection rate.

Fractions were pooled starting with the fraction when the in-line absorbance reached 5% of the observed maximal UV absorbance. After this, all fractions towards the end of the gradient with <3% dimer/aggregate as determined by SE-HPLC were made into the final pool.

### REFERENCES

The following references are herein incorporated by reference:

Ausubel, F. M., R. Brent, R. E. Kingston, D. D. Moore, J. G. Seidman, J. A. Smith and K. Struhl. 2005. “Current protocols in molecular biology”. John Wiley and Sons, New York

Balbas, P., X. Soberon, E. Merino, M. Zurita, H. Lomeli, F. Valle, N. Flores and F. Bolivar. 1986. Plasmid vector pBR322 and its special-purpose derivatives—a review. Gene 50: 3-40.

Balbas, P., X. Soberon, F. Bolivar and R. L. Rodriguez. 1988. The plasmid pBR322. In Rodriguez, R. L., and D. T. Denhardt (ed.) “Vectors. A survey of molecular cloning vectors and their uses”, pp 5-41. Butterworths, Boston, Mass.

Bogosian, G., B. N. Violand, E. J. Dorward-King, W. E. Workman, P. E. Jung and J. F. Kane. 1989. Biosynthesis and incorporation into protein of norleucine by Escherichia coli. J. Biol. Chem. 264: 531-539.

Bolivar, F., R. L. Rodriguez, P. J. Greene, M. C. Betlach, H. L. Heyneker and H. W. Boyer. 1977. Construction and characterization of new cloning vehicles. II. A multipurpose cloning system. Gene 2: 95-113.

Chasov, V. V., A. A. Deev, I. S. Masulis, and O. N. Ozoline. 2002. Distribution and functional significance of A/T tracts in promoter sequences of Escherichia coli. Mol. Biol. 36:537-542.

Khudyakov, Y. E., and H. A. Fields. 2003. “Artificial DNA: Methods and applications”. CRC Press, Boca Raton, Fla.

Lisser, S., and Margalit. 1993. Complication of E. coli mRNA promoter sequences. Nucleic Acids RES. 21:1507-1516.

Pouwels, P. H., B. E. Enger-Valk and W. J. Brammar 1985. “Cloning vectors. A laboratory manual”. Elsevier Science Publishers, Amsterdam

Sambrook, J., and D. W. Russell. 2001. “Molecular cloning. A laboratory manual”. Third Edition. Cold Spring Harbor Laboratory Press, Cold Spring Harbor, N.Y.

1. A recombinant construct comprising a ribosome binding site selected from the group consisting of SEQ ID NOs: 24-30 operably linked to a synthetic front end of a prolactin gene.

2. The recombinant construct of claim 1, wherein the ribosome binding site comprises SEQ ID NO: 24 and is operably linked to a synthetic front end of a prolactin gene comprising a sequence selected from the group consisting of SEQ ID NOs: 31-36.

3. The recombinant construct of claim 1, wherein the ribosome binding site comprises SEQ ID NO: 25 and is operably linked to a synthetic front end of a prolactin gene comprising SEQ ID NO: 37.

4. The recombinant construct of claim 1, wherein the ribosome binding site comprises SEQ ID NO: 26 and is operably linked to a synthetic front end of a prolactin gene comprising a sequence selected from the group consisting of SEQ ID NOs: 38-46.

5. The recombinant construct of claim 1, wherein the ribosome binding site comprises SEQ ID NO: 27 and is operably linked to a synthetic front end of a prolactin comprising SEQ ID NO: 47.

6. The recombinant construct of claim 1, wherein the ribosome binding site comprises SEQ ID NO: 28 and is operably linked to a synthetic front end of a prolactin comprising SEQ ID NO: 48.

7. The recombinant construct of claim 1, wherein the ribosome binding site comprises SEQ ID NO: 29 operably linked to a synthetic front end of a prolactin gene comprising a sequence selected from the group consisting of SEQ ID NOs: 49-52.

8. The recombinant construct of claim 1, wherein the ribosome binding site comprises SEQ ID NO: 30 and is operably linked to a synthetic front end of a prolactin gene comprising SEQ ID NO: 53.

9. A transformed host cell comprising the recombinant construct of claim 1 operably linked to a promoter.

10. The host cell of claim 9, wherein the host cell is a prokaryotic cell.

11. (canceled)

12. A method for producing a prolactin antagonist in a transformed host cell, the method comprising:
culturing a host cell comprising a nucleic acid sequence selected from the group consisting of SEQ ID NOs: 1-23, 24-30, 54, 55, 56, and 57 under conditions that induce gene expression from the prolactin antagonist DNA sequence.

13. The method of claim 21 wherein the synthetic front end of a prolactin gene is selected from the groups consisting of SEQ ID NOS: 31-53 and 58.

14. The method of claim 21 wherein said prolactin antagonist is the G129R variant human prolactin antagonist.

15. An isolated nucleic acid sequence comprising a sequence selected from the group consisting of SEQ ID NOs: 1-23, 54, and 55.

16. A recombinant construct comprising a ribosome binding site sequence operably linked to a prolactin antagonist DNA sequence, wherein said construct comprises a sequence selected from the group consisting of SEQ ID NOs: 1-23, 54, and 55.

17. The recombinant construct of claim 16, further defined as operably linked to a promoter functional in a host cell.

18. A transformed host cell comprising the recombinant construct of claim 17.

19. The host cell of claim 18, wherein the host cell is a prokaryotic cell.

20. (canceled)

21. The method of claim 12, wherein the nucleic acid sequence is a ribosome binding site selected from the group consisting of SEQ ID NOs: 24-30, 56, and 57 operably linked to a synthetic front end of a prolactin gene under conditions that induce gene expression from the prolactin antagonist DNA sequence.

22. The method of claim 12 wherein said prolactin antagonist is the G129R variant human prolactin antagonist.

23-32. (canceled)
